(19) United States
(12) Patent Application Publication
(10) Pub. No.: US 2022/0287922 A1
(43) Pub. Date: Sep. 15, 2022

(54) DENTIN REGENERATIVE CELL CULTURE

(71) Applicants: NATIONAL CENTER FOR GERIATRICS AND GERONTOLOGY, Obu City, Aichi (JP); SHIBUYA CORPORATION, Kanazawa-shi, Ishikawa (JP)

(72) Inventors: Misako NAKASHIMA, Obu City, Aichi (JP); Koichiro IOHARA, Obu City, Aichi (JP); Kenji YONEDA, Kanazawa-shi, Ishikawa (JP); Ryosuke MURAI, Kanazawa-shi, Ishikawa (JP); Hiroyuki NAGAI, Kanazawa-shi, Ishikawa (JP)

(21) Appl. No.: 17/633,405

(22) PCT Filed: Jul. 28, 2020
(86) PCT No.: PCT/JP2020/028838

§ 371 Date: Feb. 7, 2022

(30) Foreign Application Priority Data
Aug. 9, 2019 (JP) 2019-147175

(51) Int. Cl. A61K 6/884; A61K 35/32; A61L 27/38

(52) U.S. Cl. CPC A61K 6/884; A61K 35/32; A61L 27/38

(57) ABSTRACT

A dentin regenerative cell culture that can bring about a rapid regeneration of dentin in a deficit region. In the treatment of a tooth using the dentin regenerative cell culture, a root canal where a pulpectomy has been performed is filled with a root canal filler containing dental pulp stem cells. The dentin regenerative cell culture is then implanted in the deficit region of dentin, and temporary sealing with a packing is carried out. The dentin regenerative cell culture is formed three-dimensionally in conformity with the shape of the deficit region with the coalescence of cell masses of a plurality of odontoblasts, and thus the dentin regeneration is well promoted. In addition, gaps between the dentin regenerative cell culture and biological tissue can be rapidly filled. Infection due to bacterial infiltration can thereby be prevented.

### TECHNICAL FIELD

The present invention relates to a dentin regenerative cell culture for a tooth, and more particularly to a dentin regenerative cell culture for a tooth that has a root canal where a pulpectomy has been performed filled with a root canal filler containing dental pulp stem cells.

### BACKGROUND ART

Conventionally, as a dental treatment method, it has been proposed to fill a root canal that is irrigated after removal of the dental pulp with a root canal filler containing dental pulp stem cells, thereby allowing proliferation of the dental pulp cells to promote regeneration and functional recovery of the dental pulp and further promote differentiation of the dental pulp stem cells into odontoblasts to seal a crown portion with dentin (Patent Literature 1).

The above root canal filler has the dental pulp stem cells and an extracellular matrix (scaffold) to which the dental pulp stem cells are attached, and the root canal filler upon filling the root canal is temporarily sealed by packing with resin or the like.

In addition, as a conventional dentin regeneration method, it is known that dental pulp stem cells that are attached to an extracellular matrix in advance are cultured and differentiated into odontoblasts, and the odontoblasts from the differentiation together with the extracellular matrix are made to fill a deficit region of the dentin of a tooth (Patent Literature 2).

### PRIOR ART DOCUMENTS

### Patent Literature

Patent Literature 1: Japanese Patent No. 5621105
Patent Literature 2: International Publication No. WO2005/079728

### SUMMARY OF INVENTION

### Problems to be Solved by the Invention

In Patent Literature 1, the dental pulp stem cells filling the root canal are to be differentiated into odontoblasts to seal a deficit region of the dentin; however, regeneration of dentin takes time, and before the regenerated dentin seals the deficit region, a bacterium has sometimes infiltrated from a minute gap between the deficit region and the packing to cause bacterial infection, thereby making it impossible to regenerate dentin.

In Patent Literature 2, although the dental pulp stem cells attached to the extracellular matrix are differentiated into odontoblasts in advance, it has taken time for the extracellular matrix filling in the deficit region together with the odontoblasts to be absorbed into a living organism.

In view of such circumstances, the present invention provides a dentin regenerative cell culture that can bring about a rapid regeneration of dentin in a deficit region.

### Means for Solving the Problems

An invention of claim 1 is a dentin regenerative cell culture to be implanted in a deficit region of dentin of a tooth having a root canal filled with a root canal filler containing dental pulp stem cells, the root canal having undergone a pulpectomy, characterized in that the dentin regenerative cell culture is formed in conformity with a shape of the deficit region with coalescence of cell masses of a plurality of odontoblasts.

### Advantageous Effect of Invention

According to the invention of claim 1, the dentin regenerative cell culture is formed in conformity with the shape of the deficit region with the coalescence of the cell masses of the plurality of odontoblasts and thus well promotes the dentin regeneration when implanted in the deficit region of the dentin; in addition, the dentin regenerative cell culture has a high affinity for biological tissue and thus can rapidly and completely fill gaps between the dentin regenerative cell culture and the biological tissue, thereby preventing the bacterial infection.

### BRIEF DESCRIPTION OF DRAWINGS

FIG. 1 is an explanatory diagram of a treatment process of a tooth 1 using a dentin regenerative cell culture 4 according to the present invention.

FIG. 2 is an explanatory diagram of a process for producing the dentin regenerative cell culture 4 according to the present invention.

### MODE FOR CARRYING OUT THE INVENTION

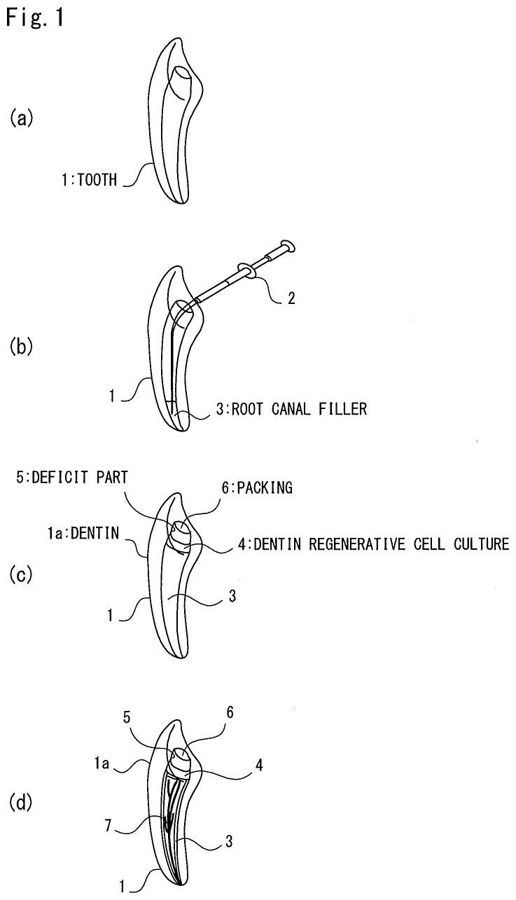

The present invention will be described below with reference to an embodiment shown in the drawings; in FIG. 1(a), reference sign 1 denotes a tooth to be treated, which has undergone pulpectomy because of bacterial infection due to dental caries, pulpitis, etc. spreading to the coronal pulp or radicular pulp.

After the pulpectomy and irrigation of a root canal portion, a root canal filler 3 in gel form is injected into the root canal of the tooth 1 on the side of its apical area using a micropipet 2, a syringe or the like as shown in FIG. 1(b).

The above root canal filler 3 is formed by attaching cells including dental pulp stem cells to an extracellular matrix (scaffold).

The above extracellular matrix is a so-called scaffold and is a scaffold for anchoring the dental pulp stem cells. The extracellular matrix in gel form is indefinite in shape, but is not limited thereto; for example, the extracellular matrix may have a definite shape such as a cylindrical shape or a substantially conical shape to facilitate filling in the root canal. When the root canal filler 3 has a definite shape, forceps or the like may be used to fill in the apical area side of the root canal with the root canal filler 3.

The extracellular matrix constituting the above root canal filler 3 preferably includes a living organism affinitive material containing at least one of collagen, artificial proteoglycan, gelatin, hydro-gel, fibrin, phosphophoryn, heparan sulphate, heparin, laminin, fibronectin, alginate, hyaluronic acid, chitin, polylactic acid (PLA), poly(lactic-co-glycolic acid) (PLGA), polyethylene glycol (PEG), polyglycolic acid (PGA), poly-DL-lactic acid (PDLLA), polycaprolactone (PCL), hydroxy apatite, β-TCP, calcium carbonate, titanium, and gold. Note that proteoglycan is a type of glycoconjugate in which a protein and a sugar chain (glycosaminoglycan) are covalently linked with each other.

The collagen used as the above extracellular matrix is preferably a mixed collagen that is a mixture of type I collagen and type III collagen. Type I collagen is a basic collagen and is fibrillar collagen. Type III collagen forms a thin mesh structure called reticular fiber, which is different from collagen fibers, and makes a scaffold of cells or the like.

The dental pulp stem cells constituting the above root canal filler 3 are dental pulp stem cells derived from a wisdom tooth, permanent dentition or deciduous tooth, and may be autologous cells extracted from a patient who will undergo a tooth-tissue regeneration treatment or may be allogeneic cells extracted from a person other than the patient who will undergo the tooth-tissue regeneration treatment.

After the root canal filler 3 is injected into the apical area side of the root canal of the above tooth 1, as shown in FIG. 1(c), the dentin regenerative cell culture 4 according to the present invention is implanted in a deficit region 5 of dentin la of the tooth 1. The above deficit region 5 is temporarily sealed by a packing 6 such as resin outside the dentin regenerative cell culture 4.

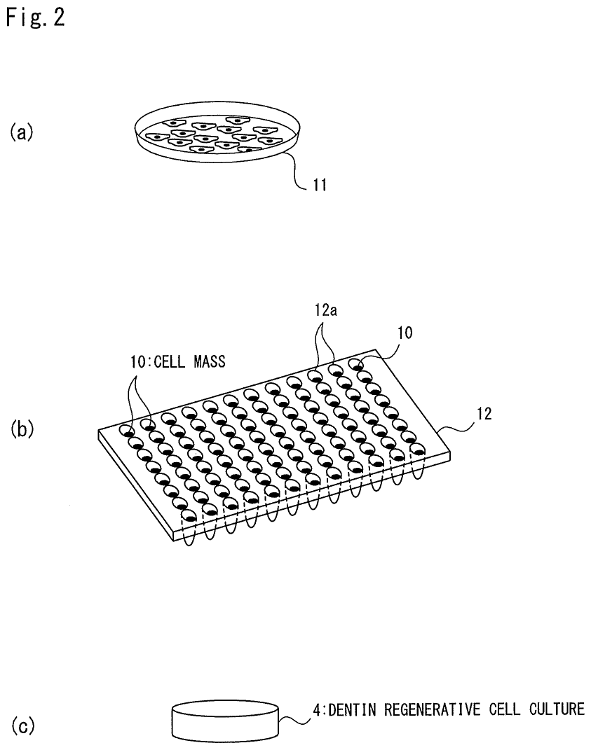

The above dentin regenerative cell culture 4 is formed three-dimensionally in conformity with a shape of the deficit region 5 with coalescence of cell masses 10 (see FIG. 2) of a plurality of odontoblasts.

FIG. 2 is a process diagram showing a producing process of the above dentin regenerative cell culture 4; as shown in FIG. 2(a), first, autologous dental pulp stem cells collected from an unnecessary tooth such as a wisdom tooth are cultured in a dish 11. Next, as shown in FIG. 2(b), the dental pulp stem cells cultured in the dish 11 are seeded in each well 12a of a well plate 12 to prepare a cell mass 10. The cell mass 10 is a substantially spherical cell aggregation in which cells are clustered and aggregated and can be easily and efficiently prepared by culturing cells in the above each well 12a having a substantially hemispherical container whose surface is non-adherent to cells. In the preparation of the cell mass 10, 100 μl of a culture solution and 7000 to 20000 cells can be placed in each well 12a and cultured until the diameters become about 300 to 500 μm; more preferably, about 15000 cells resulting in a diameter of about 400 μm allow the culture solution to spread to the center of the cell mass and facilitate handling for forming into a desired shape.

Afterward, a plurality of grown cell masses 10 are three-dimensionally stacked in contact with each other in conformity with a shape of the above deficit region 5 by a conventionally known 3D bioprinter or the like (not shown) disclosed in Japanese Patent No. 5896104, for example, and as shown in FIG. 2(c), these cell masses 10 three-dimensionally stacked are coalesced together to prepare the above dentin regenerative cell culture 4 having a desired shape.

In this case, in a state of the above cell masses 10 or in a state in which the dentin regenerative cell culture 4 having the desired shape is prepared, a growth factor (BMP) is added at an increased concentration of phosphate to induce differentiation of the dental pulp stem cells into odontoblasts. Alternatively, the cell mass 10 may be prepared after inducing the differentiation of dental pulp stem cells into odontoblasts in the dish 11.

When the above tooth 1 is an anterior tooth (with a single root canal), a typical shape of the deficit region 5 is a substantially cylindrical recess; in this case, a shape of the dentin regenerative cell culture 4 is, for example, a cylindrical shape having a diameter of from 0.7 to 1.0 mm and a height of about 0.3 mm (see FIG. 2(c)).

When the above tooth 1 is a molar tooth (with multiple root canals), though not shown, the shape of the deficit region 5 is a plurality of substantially cylindrical recesses or a single recess having a complex shape in which a plurality of substantially cylindrical recesses are connected to each other; thus, the dentin regenerative cell culture 4 produced will take a shape according to the shape of the deficit region.

In any case, it is desirable that the dentin regenerative cell culture 4 is formed slightly larger than the shape of the deficit region 5, and by press-fitting the dentin regenerative cell culture 4 into the deficit region 5 through the elasticity of the dentin regenerative cell culture, the inner surface of the deficit region 5 and the outer surface of the dentin regenerative cell culture 4 can be brought into close contact with each other.

In the tooth 1 treated in a manner described above, the above root canal filler 3 regenerates dental tissue in the root canal. As shown in FIG. 1(d), the regenerated tooth tissue is, for example, a blood vessel 7 and pulp tissue in the root canal. On the other hand, since the dentin regenerative cell culture 4 is formed with coalescence of the cell masses 10 of the plurality of odontoblasts, the regeneration of dentin is well promoted. In addition, since the dentin regenerative cell culture 4 contains an extracellular matrix such as collagen secreted from the odontoblast, has a high affinity for the root canal filler and biological tissue due to the action of a growth factor contained in the extracellular matrix, and is formed in conformity with the shape of the above deficit region 5, the gaps between the dentin regenerative cell culture 4 and the biological tissue can be completely filled rapidly, thereby making it possible to prevent infection due to bacterial infiltration.

Note that the tooth 1 to be treated may be either the tooth 1 that has not been extracted or the tooth 1 that has been extracted, and the extracted tooth 1 would be re-implanted into a socket.

In addition, when the dentin regenerative cell culture is prepared from allogeneic cells, it is only required to induce differentiation of the dental pulp stem cells into odontoblasts, followed by a decellularization treatment, which can suppress immunorejection.

### REFERENCE SIGNS LIST

1 Tooth
1a Dentin
3 Root canal filler
4 Dentin regenerative cell culture
5 Deficit part
6 Packing
10 Cell mass

1. A dentin regenerative cell culture to be implanted in a deficit region of dentin of a tooth having a root canal filled with a root canal filler containing dental pulp stem cells, the root canal having undergone a pulpectomy, wherein
the dentin regenerative cell culture is formed in conformity with a shape of the deficit region with coalescence of cell masses of a plurality of odontoblasts.

2. The dentin regenerative cell culture according to claim 1, wherein the odontoblasts are obtained by inducing differentiation of dental pulp stem cells.

3. The dentin regenerative cell culture according to claim 2, wherein the odontoblasts are obtained by inducing differentiation of dental pulp stem cells, followed by a decellularization treatment.
